(19) United States
(12) Patent Application Publication
(10) Pub. No.: US 2024/0245049 A1
(43) Pub. Date: Jul. 25, 2024

(54) CELLULAR TRANSPLANT CARRIER DEVICES AND METHODS OF USING THE SAME

(71) Applicant: THE REGENTS OF THE UNIVERSITY OF COLORADO, A BODY CORPORATE, Denver, CO (US)

(72) Inventors: Marc MATHIAS, Denver, CO (US); Kang LI, Baltimore, MD (US); Miguel FLORES BELLVER, Centennial, CO (US); Silvia APARICIO DOMINGO, Centennial, CO (US); Conan CHEN, Denver, CO (US); Valeria CANTO-SOLER, Denver, CO (US)

(73) Assignee: THE REGENTS OF THE UNIVERSITY OF COLORADO, A BODY CORPORATE, Denver, CO (US)

(21) Appl. No.: 18/627,309

(22) Filed: Apr. 4, 2024

Related U.S. Application Data
(63) Continuation of application No. PCT/US2022/045569, filed on Oct. 3, 2022.
(60) Provisional application No. 63/252,134, filed on Oct. 4, 2021.
(60) Provisional application No. 63/280,974, filed on Nov. 18, 2021.

(51) Int. Cl. A01N 1/02; A61F 9/00; A61K 35/30

(52) U.S. Cl. CPC A01N 1/0263; A61F 9/0017; A61K 35/30

(57) ABSTRACT

A cell transplant carrier including a carrier plate and a carrier housing. The carrier plate includes a body and one or more chambers defined in the body. Each of the one or more chambers is configured to receive a cell transplant and is shaped to cause the cell transplant to be at least partially exposed in order to permit removal of the cell transplant therefrom. The carrier housing includes a carrier plate opening leading to an inner compartment configured to removably receive the carrier plate.

### CROSS-REFERENCE TO RELATED APPLICATIONS

This application is a continuation application of International Application No. PCT/US2022/045569, filed Oct. 3, 2022, which claims priority under 35 U.S.C. § 119(e) to U.S. Provisional Application No. 63/252,134, filed Oct. 4, 2021, and U.S. Provisional Application No. 63/280,974, filed Nov. 18, 2021. These applications are incorporated herein by reference in their entireties for all purposes.

### FIELD

Embodiments of the instant disclosure relate to devices and methods of harvesting, preserving, and transporting cellular implantations to an eye of a subject in need thereof.

### BACKGROUND

Retinal pigment epithelium (RPE) plays an important role in supporting normal photoreceptor function. RPE damage can lead to secondary dysfunction and degeneration of photoreceptor cells, which in turn can cause severe, irreversible vision impairment in patients affected by conditions such as age-related macular degeneration (AMD) and Stargardt's disease. Recently, clinical trials involving transplantation of embryonic stem cell-derived RPE in patients with AMD showed promising safety and efficacy outcomes. Moreover, RPE derived from human-induced pluripotent stem cells (hiPSC), which could be utilized for autologous therapies, is also being evaluated in clinical trials. Accordingly, subretinal delivery devices for preservation of harvested or cultured cells such as stem cells and preservation and delivery of RPE single cells and/or monolayers of RPE cells or tissue are needed in this advancing field.

### SUMMARY

Embodiments of the instant invention concern cellular implant carrier devices. In certain embodiments, a cell transplant carrier, can include one or more drawer having one or more chambers, wherein each of the one or more chambers can be configured to receive a cell transplant having a tab that allows removal of the cell transplant from the chamber; and a chest including one or more slots configured to removably receive one or more drawers. In embodiment, at least one of the one or more drawer and the one or more slot are rectangular in shape.

In other embodiments, the carrier can include a chest having a cuboid shape. In some embodiments, the carrier device having one or more chambers can be symmetrically distributed along the dimensions of the drawer.

In other embodiments, a cell transplant carrier can further include one or more tubes configured to receive the cell transplant carrier. In certain embodiments, the tube can be configured to further include a stabilizing or media capable of preserving the cells or tissues to reduce degradation and/or contamination, for example.

In other embodiments, methods for stabilizing or preserving the viability of cells or a cellular implant or cellular tissue are described. In accordance with these embodiments, stabilizing or preserving the viability of cells or cellular implant or cellular tissue can include placing a cell, the cells, a cellular implant or cellular tissue into a cell transplant carrier disclosed herein, transferring the cell transplant carrier into a tube configured to receive the cell transplant carrier to form a carrier device; filling the carrier device with a medium and preserving the viability of the cell, the cells or cellular implant or cellular tissue. In certain embodiments, the medium includes a medium for stabilizing stem cells or other cells in order reduce degradation and maintain viability.

Other embodiments concern kits containing devices and kits for transporting and preserving harvested, reprogrammed, or differentiated cells or tissues.

Some embodiments concern using devices disclosed herein in conjunction with or in addition to, surgical delivery devices to treat an eye condition in a subject. Some embodiments concern treating a condition of the retina.

Embodiments of the instant invention can include devices having a cell carrier including a carrier plate and a carrier housing. In accordance with these embodiments, the carrier plate can include a body and one or more chambers defined in the body. Each of the one or more chambers can receive a cell transplant therein. In certain embodiments, the cell transplant as referenced herein includes or is a cell-containing substance, for example, a stem cell population (e.g. present in multiple or in a monolayer), a differentiated cell population or tissue containing cells of use for implantation in a subject, or a substrate having a plurality of cells on the substrate. In some embodiments, the carrier housing can include a carrier plate opening leading to an inner compartment that can removably receive the carrier plate.

In certain embodiments, the carrier plate is rectangular in shape.

In certain embodiments, each of the one or more chambers can include a slot portion defining an overhang of the body of the carrier plate, the slot portion configured to retain the cell transplant reducing or eliminating migration therefrom.

In certain embodiments, each of the one or more chambers is positioned on a side edge of the carrier plate. In certain embodiments, each of the one or more chambers can be recessed into the side edge of the carrier plate.

In certain embodiments, each of the one or more chambers includes a recessed portion defined on the side edge of the carrier plate, the cell transplant including a tab portion configured to overhang the recessed portion when the cell transplantis received within the one or more chambers.

In certain embodiments, the one or more chambers are symmetrically distributed along the dimensions of the drawer.

In certain embodiments, the carrier housing can include a plurality of openings extending from an outer surface to the inner compartment to provide a passageway for a preservation media to pass therethrough.

In certain embodiments, the carrier housing can include four sidewalls, the plurality of passageways extending through at least two of the sidewalls.

In certain embodiments, in an assembled state, the carrier plate is positioned within the inner compartment of the carrier housing and the tab portion of the cell transplant opposes one of the sidewalls of the carrier housing in which the plurality of passageways do not extend there through.

In certain embodiments, the carrier housing, and the carrier plate, when positioned within the inner compartment of the carrier housing, are sized to fit within a 2.0 mL tube volume for storage and/or transport.

In certain embodiments, each of the one or more chambers can include two symmetrical flat tabs configured to hold the cell transplant in place in a correct or fixed orientation within each chamber.

In certain embodiments, at least one of the one or more chambers has a length of about 2 mm to about 20 mm, a width of at least 1 mm and not more than 3 mm, and a height of at least 0.25 mm and not more than 0.75 mm.

In certain embodiments, the carrier housing has a cuboid shape.

In certain embodiments, the carrier housing and/or carrier plate is 3D printed.

In certain embodiments, the carrier housing and/or the carrier plate is manufactured from at least one of VeroClear, Med610, Med615, and Med620.

In certain embodiments, the cell transplant includes a retinal pigment epithelium (RPE) cell transplant. In accordance with these embodiments, the retinal pigment epithelium cell transplant includes a plurality of cells in one or more layers or on a substrate. In certain embodiments, each cell transplant can include between about 5,000 and about 20,000 cells. In certain embodiments, each cell transplant can include between about 10,000 and about 20,000 cells. In certain embodiments, each cell transplant can include between about 10,000 and about 15,000 cells.

In certain embodiments, the cell transplant includes a neural retina-RPE cellular transplant. In accordance with these embodiments, the neural retina-RPE transplant includes a plurality of cells. In certain embodiments, each cell transplant can include between about 5,000 and about 20,000 cells. In certain embodiments, each cell transplant can include between about 10,000 and about 20,000 cells. In certain embodiments, each cell transplant can include between about 10,000 and about 15,000 cells.

In certain embodiments, the cell transplant includes retinal cells. In accordance with these embodiments, the retinal cells includes a plurality of cells. In certain embodiments, each cell transplant can include between about 5,000 and about 20,000 cells. In certain embodiments, each cell transplant can include between about 10,000 and about 20,000 cells. In certain embodiments, each cell transplant can include between about 10,000 and about 15,000 cells.

In certain embodiments, each of the one or more chambers is shaped to cause a portion of the cell transplant to be at least partially exposed to permit removal of the cell transplant therefrom.

Embodiments of the instant invention can include a carrier device including a cell transplant cell carrier as described herein, and one or more cell transplants housed within the cell carrier, and a chamber configured to receive the carrier device therein.

In certain embodiments, the carrier device can further include a medium that can be received within the chamber. In accordance with these embodiments, the medium can include any cell culture medium, storage medium, stem cell medium or the like.

In certain embodiments, the carrier device can further include a temperature controller configured to control temperature of the medium and/or cell transplant.

In certain embodiments, the carrier device can further include an oxygen controller configured to control oxygen levels of the medium and/or cell transplant.

In certain embodiments, the carrier device can further include a carbon dioxide (C02)controller configured to control pH of the medium and/or cell transplant. In some embodiments, the pH range can be from about 6.5 to about 8.5.

Embodiments of the instant invention can include methods of preserving viability of cells or a cellular implant or cellular tissue or a single cell transplant. In accordance with these embodiment, methods can include placing the cells or the cellular implant or the cellular tissue or the cell into a cell transplant carrier according to the present disclosure. The methods can further include transferring the cell transplant carrier into a chamber configured to receive the cell transplant carrier to form a carrier device. In addition, the methods can further include filling the carrier device with a medium and preserving the viability of the cell, cells or cellular implant or cellular tissue.

In certain embodiments, methods can further include removing the cell, cells or the cellular implant or cellular tissue from the cell transplant carrier using a surgical device.

In certain embodiments, methods can further include implanting the cell, the cells or the cellular implant or cellular tissue into an eye of a subject using the carrier device and treating an eye condition in the subject.

In certain embodiments, the eye condition can include a condition of the retina.

Embodiments of the instant invention can include a kit including a cell carrier as described herein. In accordance with these embodiments, the kit can further include one or more cell transplants housed within the cell carrier, and instructions for use.

In certain embodiments, the kit can further include a trephine configured to cut the cell transplant. In certain embodiments, the trephine can be configured to cut the cell transplant in a shape. In some embodiments the shape can include a circular portion and a tab portion extending outward from a perimeter of the circular portion.

In certain embodiments, the kit can include a surgical implantation device configured to releasably grasp the cell transplant.

### BRIEF DESCRIPTION OF THE FIGURES

FIG. 1A illustrates a retinal pigment epithelium cell spheroid derived from retinal organoids.

FIG. 1B represents an exemplary photographic image demonstrating characteristic pigmentation, hexagonal morphology, and intact F-actin cytoskeleton of the RPE cells in culture of certain embodiments disclosed herein.

FIG. 1C represents an exemplary fluorescent image demonstrating intact F-actin cytoskeleton (Phalloidin) and proper localization of premelanosome protein (PMEL17) in hiPSC-derived RPE monolayers in culture of certain embodiments disclosed herein.

FIG. 1D represents an exemplary fluorescent image demonstrating a healthy and functionally mature state of experimental tissue. Certain markers including orthodenticle homeobox 2 (OTX2), and zonula occludens-1 (ZO-1) also demonstrated normal patterns of expression in monolayers in culture of certain embodiments disclosed herein.

FIG. 2A is a block diagram of exemplary steps in preparing, storing, transporting, and implanting a subretinatl implant into a patient eye of certain embodiments disclosed herein.

FIG. 2B represents an image of a side view of a trephine having a distal cutting tip that outlines the shape of the cell transplant of certain embodiments disclosed herein.

FIG. 2C is a top view of a cell transplant, the representative shape of which is defined by the cut made by the distal cutting tip of the trephine of FIG. 2B of certain embodiments disclosed herein.

FIG. 2D are isometric top views of a cell carrier and its component pieces, a carrier housing or chest and a carrier plate or drawer of certain embodiments disclosed herein.

FIG. 2E is a close-up top view of a chamber of the carrier plate with a cell transplant, illustrated in dotted line, positioned within the chamber of certain embodiments disclosed herein.

FIGS. 2F-2M illustrate, respectively, an isometric top view, an isometric side view, a chamber side view, an opposite side view, a first end view, a second end view, a bottom view, and a top view of the carrier plate of certain embodiments disclosed herein.

FIGS. 2N-2U depict, respectively, a first isometric top view, a second isometric top view from an opposite side, a side view, an opposite side view, a first end view, a second end view, a top view, and a bottom view of the carrier housing of certain embodiments disclosed herein.

FIG. 3 illustrates the cell carrier positioned in a chamber such as a sealable tube.

FIG. 4A represents an isometric view of an exemplary surgical implantation device for retrieving a cell transplant from a carrier plate of a cell carrier of certain embodiments disclosed herein.

FIG. 4B represents an enlarged cross-sectional side view of an implantation device of certain embodiments disclosed herein.

FIGS. 4C-4F illustrate subsequent steps of grasping of a cell transplant by the forceps of the implantation device and the housing of the cell transplant in the lumen of the tubular sheath for use in a surgical procedure in accordance with certain embodiments disclosed herein.

### DETAILED DESCRIPTION

Embodiments of the present disclosure provides devices for harvesting, storing and transporting cell, cells and cellular tissue for delivery to an eye of a subject.

It is noted that as used herein and in the appended claims, the singular forms “a”, “an”, and “the” include plural reference unless the context clearly dictates otherwise. Thus, for example, reference to “an appliance” includes a plurality of such appliances and equivalents thereof known to those skilled in the art, and so forth. As well, the terms “a” (or “an”), “one or more” and “at least one” can be used interchangeably herein. It is also to be noted that the terms “comprising”, “including”, and “having” can be used interchangeably.

Number ranges are to be understood as inclusive, i.e. including the indicated lower and upper limits. Furthermore, the term “about”, as used herein, and unless clearly indicated otherwise, generally refers to and encompasses plus or minus 10% of the indicated numerical value(s). For example, “about 10%” may indicate a range of 9% to 11%, and “about 1” may include the range 0.9-1.1.

The term “biocompatible,” as used herein, refers to a material that does not elicit an immunological rejection or detrimental effect, referred herein as an adverse immune response when it is disposed within an in-vivo biological environment. For example, in embodiments a biological marker indicative of an immune response changes less than 10%, or less than 20%, or less than 25%, or less than 40%, or less than 50% from a baseline value when a human or animal is exposed to or in contact with the biocompatible material. Alternatively, immune response may be determined histologically, wherein localized immune response is assessed by visually assessing markers, including immune cells or markers that are involved in the immune response pathway, in and adjacent to the material. In one embodiment, a biocompatible material or device does not observably change immune response as determined histologically. In some embodiments, the disclosure provides biocompatible devices configured for long-term use, such as on the order of weeks to months, without invoking an adverse immune response in the subject. Biological effects can be initially evaluated by measurement of cytotoxicity, sensitization, irritation and intracutaneous reactivity, acute systemic toxicity, pyrogenicity, subacute/subchronic toxicity and/or implantation. Biological tests for supplemental evaluation can include testing for chronic toxicity.

“Bioinert” refers to a material that does not elicit an immune response from a human or animal when it is disposed within an in-vivo biological environment. For example, a biological marker indicative of an immune response remains substantially constant (plus or minus 5% of a baseline value) when a human or animal is exposed to or in contact with the bioinert material. In some embodiments, the disclosure provides bioinert devices.

Embodiments of the instant invention concern cellular implant carrier devices. In some embodiments, the implant carrier devices, in addition to the biocompatible and bioinert definitions provided herein, include material for the devices that does not cause toxicity to the cells/tissues being stored and transported (e.g., does not cause the cells to die, or trigger abnormal cellular functions, etc). This also applies to implantation devices disclosed herein. These devices are manufactured from materials that do not have any adverse/toxic effect on the cells or cellular transplants being delivered or while being manipulated within the implant device. In certain embodiments, the material of manufacture is clear or at least opaque in order to visually observe the contents (e.g., cellular substrate) of the carrier.

In certain embodiments, a cell transplant carrier, can include one or more drawer having one or more chambers, wherein each of the one or more chambers can be configured to receive a cell transplant having at least one tab. In accordance with these embodiments, the at least one tab allows removal of the cell transplant from the chamber; and a chest including a slot configured to removably receive the drawer. In certain embodiments, the tab provides a surface area exposed outside of the chamber in which the cellular transplant can be grabbed and removed from the chamber. In embodiment, the one or more drawer and the one or more slot can be rectangular in shape.

In other embodiments, the carrier can include a chest having a cuboid shape. In some embodiments, the carrier device having one or more chambers includes a feature where the one or more chambers can be symmetrically distributed along the dimensions of the drawer. In other embodiments, the chest has a length of about 10 mm to about 40 mm, a width of about 2 mm to about 20 mm, and a height of about 3 to about 10 mm. In other embodiments, devices disclosed herein include at least one but no more than 10 chambers or at least 3 chambers or no more than 7 chambers. In other embodiments, chambers can be symmetrically distributed along the dimensions of the drawer. In yet other embodiments, the at least one of the one or more chambers has a length of about 2 mm to about 20 mm, a width about 1 mm to about 3 mm, and a height of about 0.25 mm to about 0.75 mm. In yet other embodiments, volume of a cellular implant or cell transplant or cells inside a chamber makes up about 50%, 60%, 70%, or about 80% of the volume of the chamber. In yet other embodiments, each of the one or more chambers includes two symmetrical flat tabs configured to hold cells or a cellular substrate or the cellular implant or the cellular transplant in place in a correct orientation for storage and transportation of the carrier and cells or cell transplant therein.

In other embodiments, one or more of the devices disclosed herein can include one or more through holes in its top or bottom wall configured to allow a solution to be in close proximity or in contact with the cellular substrate or cellular implant or cell.

In other embodiments, the carrier of devices disclosed herein can be 3D printed. In certain embodiments, the carrier can be printed using any biocompatible 3D printing material. In some embodiments, a biocompatible 3D printing material does not have an adverse/toxic effect on the cellular substrate, cellular implant or cellular transplant. In certain embodiments, the biocompatible 3D printing material can be any biocompatible polymer material. In some embodiments, a biocompatible 3D printing material does not have an adverse/toxic effect on the cellular substrate, cellular implant or cellular transplant or the subject receiving such an implant, substrate, or transplant. In other embodiments, a 3D printing material can include a polymer such as commercially available polymers including VeroClear, and biocompatible 3D printing polymeric materials such as Med610, Med615, and Med620 or other material. In certain embodiments, the cell implant, cellular substrate or cellular transplant includes retinal pigment epithelium (RPE) cell transplant or comparable or similar cells thereof.

In other embodiments, a cell transplant carrier can further include one or more tubes configured to receive the cell transplant carrier. In certain embodiments, the tube can be configured to further include a stabilizing composition or media capable of preserving the cells or tissues to reduce degradation, preserve viability, and/or reduce or eliminate contamination, for example. In accordance with these embodiments, a stabilizing composition or media can be one that preserves the cells and is compatible with implantation or transplantation into a subject. In other embodiments, the tube can have a total volume of about 1 ml to about 4 mls. In other embodiments, cellular transplant carriers can be configured to preserve the viability of at least 80%, 90%, or 95% of the cells of a cell transplant, implant or substrate for up to or about 2 hours, about 4 hours, about 8 hours, about 12 hours, about 16 hours, about 24 hours, about 36 hours, or for about 48 hours or more.

In other embodiments, methods for stabilizing or preserving the viability of cells or a cellular implant or cellular tissue or cellular substrate or cellular transplant are described. In accordance with these embodiments, stabilizing or preserving the viability of cells or cellular implant or cellular tissue or cellular substrate or cellular transplant can include placing the cells or a cellular implant or cellular tissue or cellular substrate or cellular transplant into a cell transplant carrier disclosed herein, transferring the cell transplant carrier into a tube configured to receive the cell transplant carrier to form a carrier device; filling the carrier device with a stabilizing composition or medium and preserving the viability of the cells or cellular implant or cellular tissue or cellular substrate or cellular transplant. In other embodiments, methods disclosed herein include use of the carrier device to store, preserve, and transport at least about 80%, at least about 90%, or at least about 95% up to 100% viable cells of the cell transplant or cell implant to the site of implantation or transplantation. Other embodiments concern removing the cells or the cellular implant, cellular transplant, cellular substrate or cellular tissue from the cell transplant carrier using a surgical device.

In yet other embodiments, surgical devices disclosed herein can be used for implanting or transplanting the cells or the cellular implant or cellular tissue into an eye of a subject using the surgical device in conjunction with cellular carrier and storage devices disclosed herein and treating an eye condition in the subject. In some embodiments, an eye condition of a subject to be treated can include any eye condition in need of cellular implantation or transplantation. In other embodiments, an eye condition contemplated herein can include at least one condition of the retina.

Other embodiments disclosed herein concern kits for storing or transporting devices and/or for transporting and preserving harvested, reprogrammed, or differentiated cells or tissues. In certain embodiments, kits include devices for storing the cells or cellular implants or cellular transplants and the cells, cellular implants and cellular transplants for immediate use.

Some embodiments concern using devices disclosed herein in conjunction with surgical delivery devices to treat an eye condition in a subject. Some embodiments concern treating at least one condition of the retina.

All publications, patents, and patent applications mentioned in this specification are herein incorporated by reference to the same extent as if each individual publication, patent, or patent application was specifically and individually indicated to be incorporated by reference.

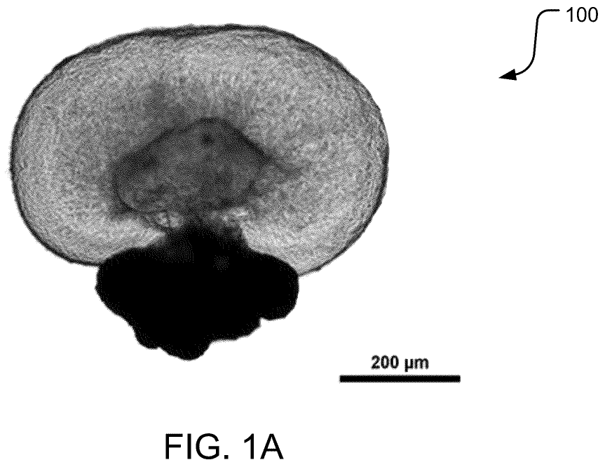

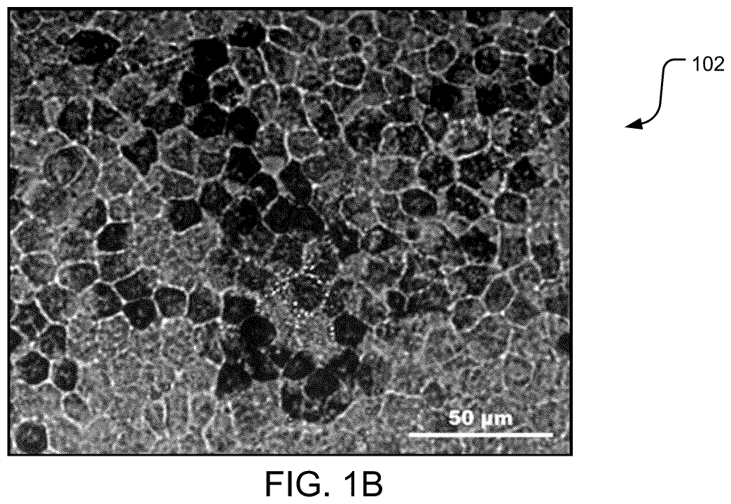

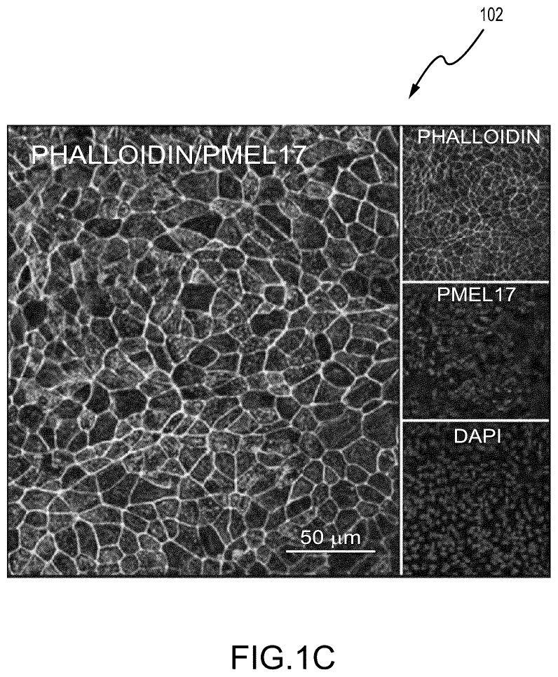

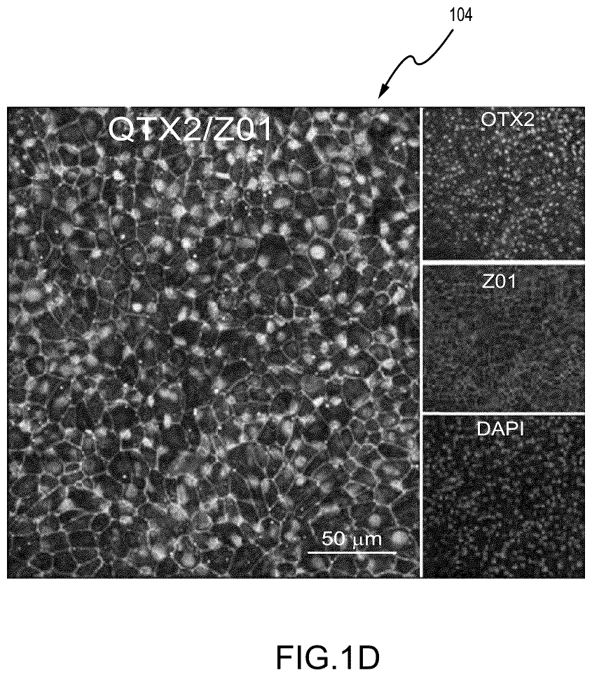

With reference to FIG. 1A, which illustrates retinal pigment epithelium (RPE) spheroids from retinal organoids 100, an embodiment of the present disclosure can include generating induced pluripotent stem cells (e.g., human induced PSCs (hiPSC)) derived from RPE monolayers to use as a cellular implant (e.g., RPE cellular implant). The RPE spheroids from the retinal organoids of FIG. 1A can be harvested and cultured on, for example, polyester membranes (e.g., 10 μm thick transparent polyester membranes). After a period of time of days to over a month (e.g., 40 days of differentiation), hiPSC-RPE cells 102 demonstrated characteristic pigmentation, typical hexagonal morphology, and intact F-actin cytoskeleton, as illustrated in FIGS. 1B-1C.

In certain embodiments, hiPSC-RPE cells can also be characterized by the expression of key proteins involved in normal RPE cell differentiation and function, including premelanosome protein (PMEL17), orthodenticle homeobox 2 (OTX2), and zonula occludens-1 (ZO-1). While PMEL17 is known to be enriched in premelanosomes, OTX2 aids in differentiation of RPE cells and transactivation of the genes involved in melanosome formation, and ZO-1 is a membrane-associated tight junction adaptor protein that links junctional membrane proteins to the cytoskeleton and plays an important role in RPE homeostasis in vivo. In accordance with these embodiments, expression of PMEL17, OTX2, and ZO-1 in these cell populations confirms that hiPSC-RPE tissue 104 is in a healthy and functionally mature state, as represented in FIG. 1D.

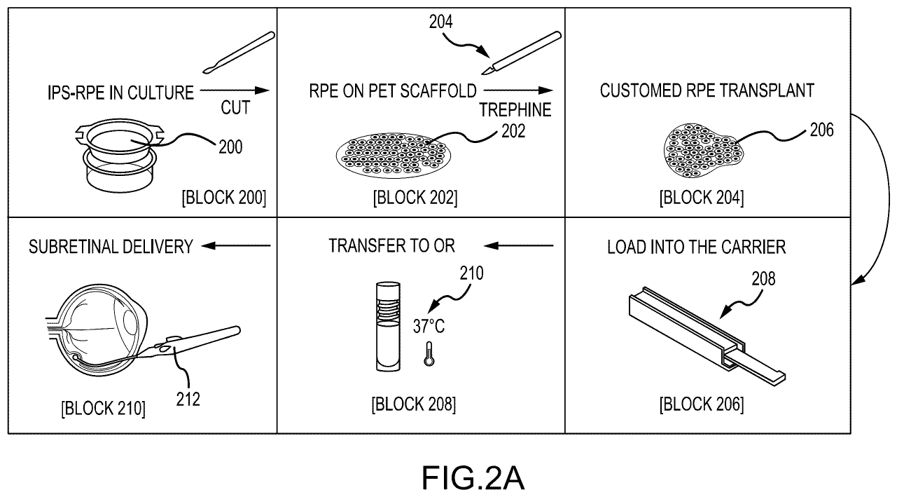

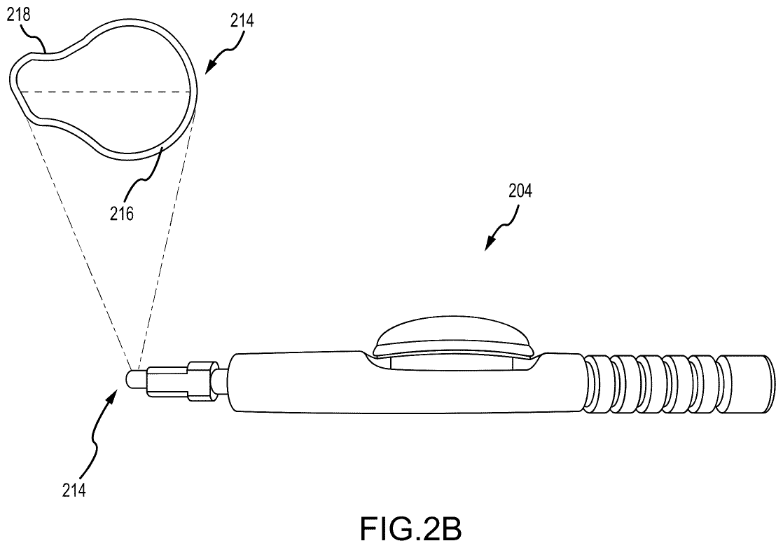

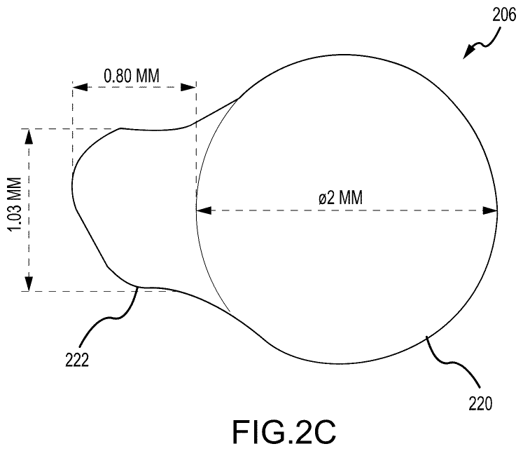

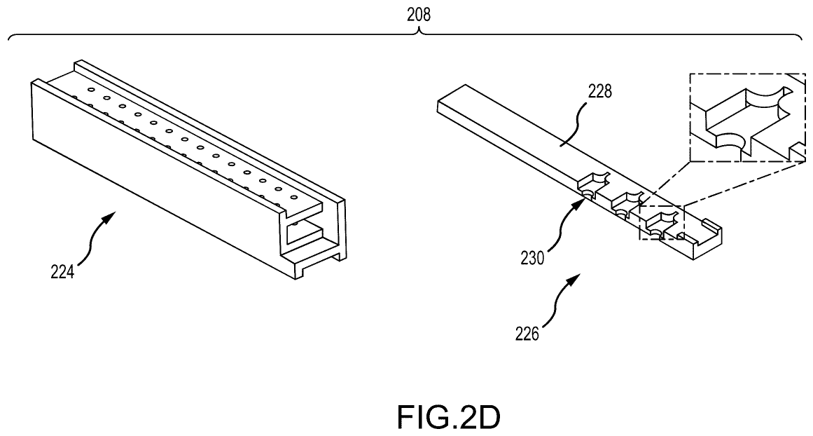

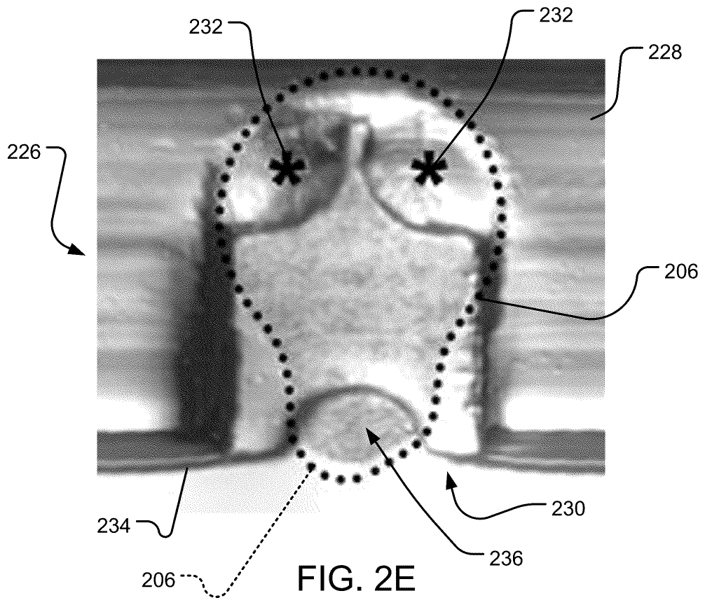

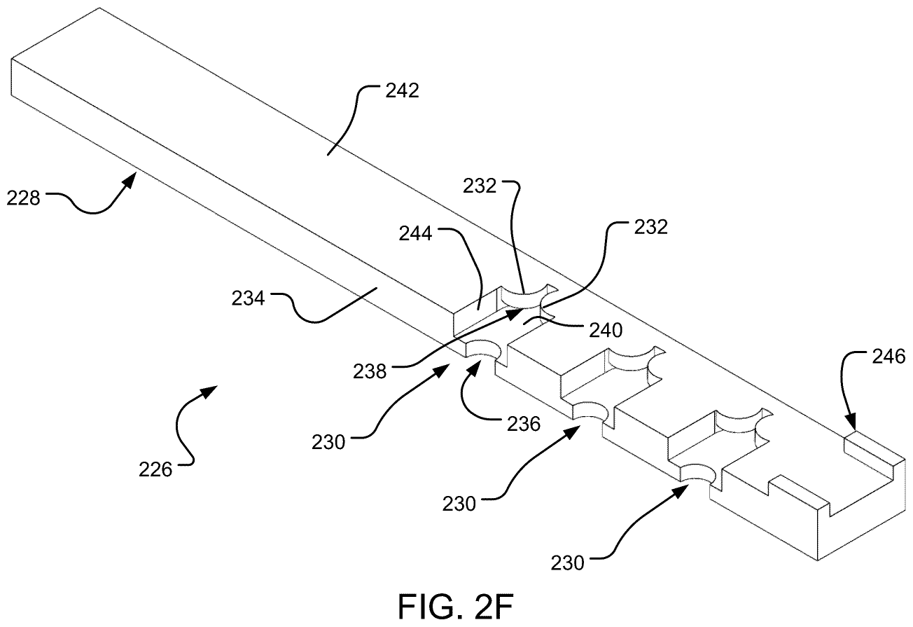

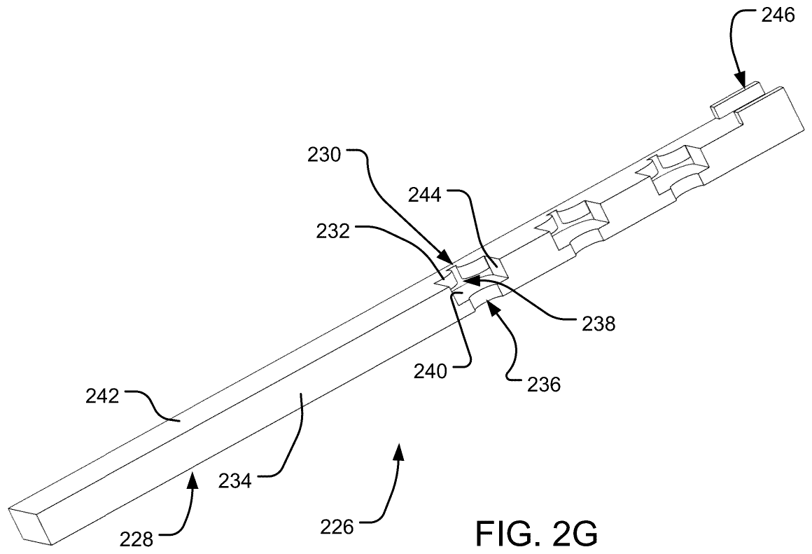

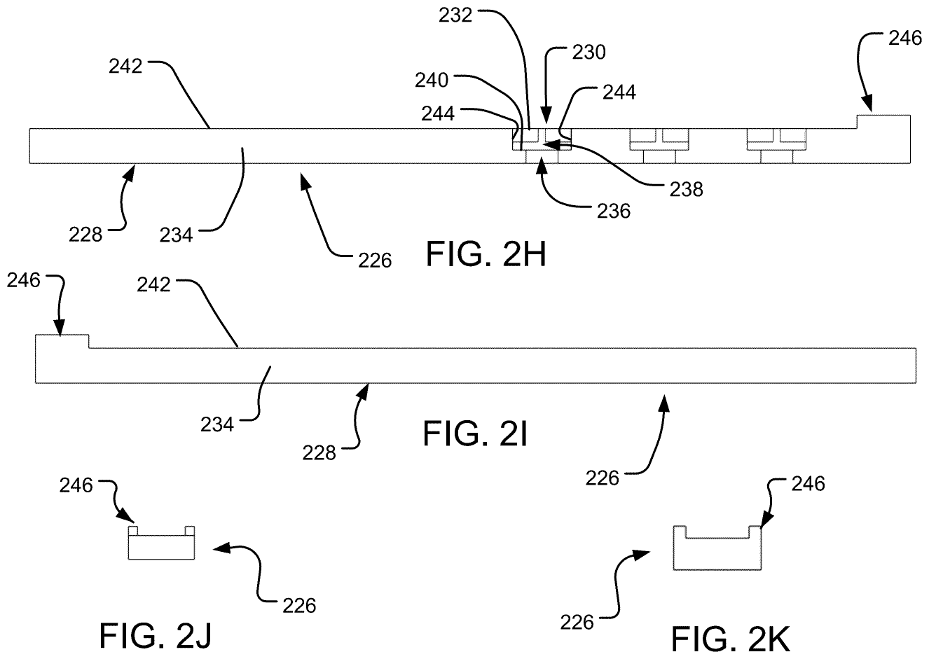

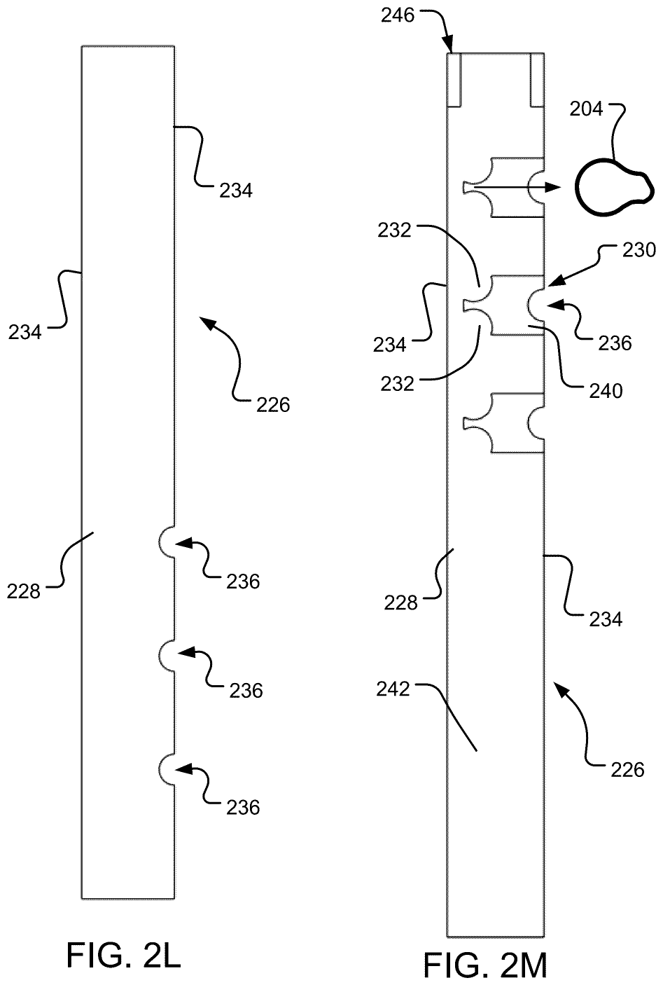

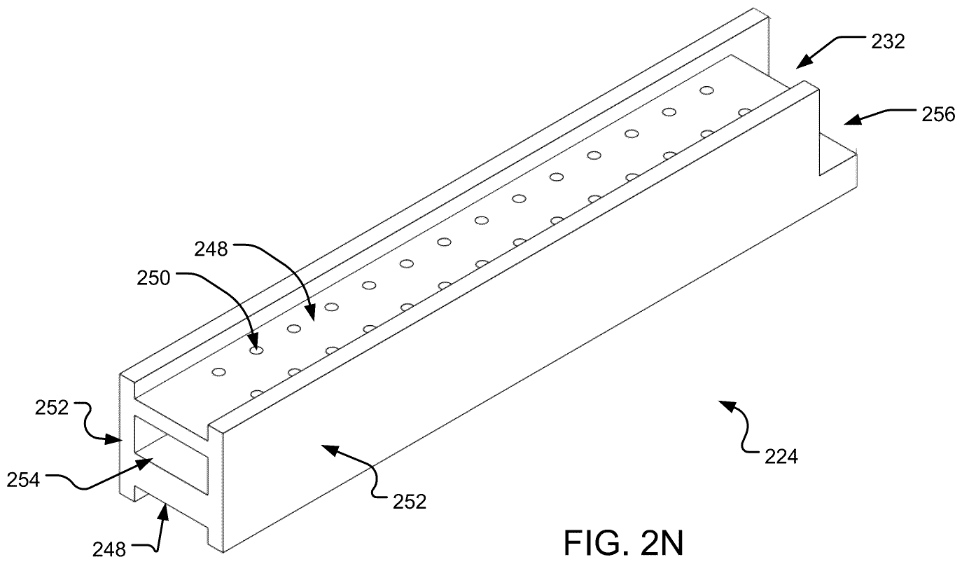

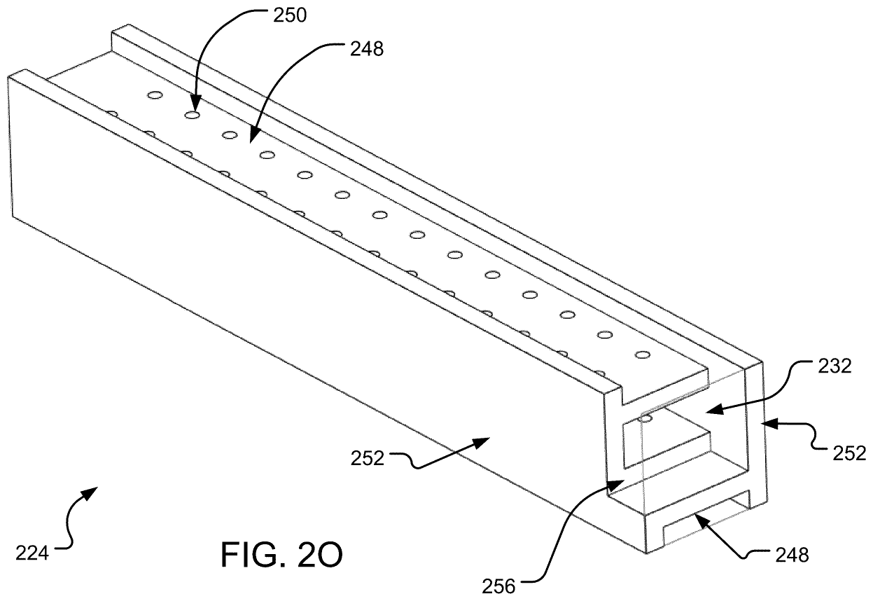

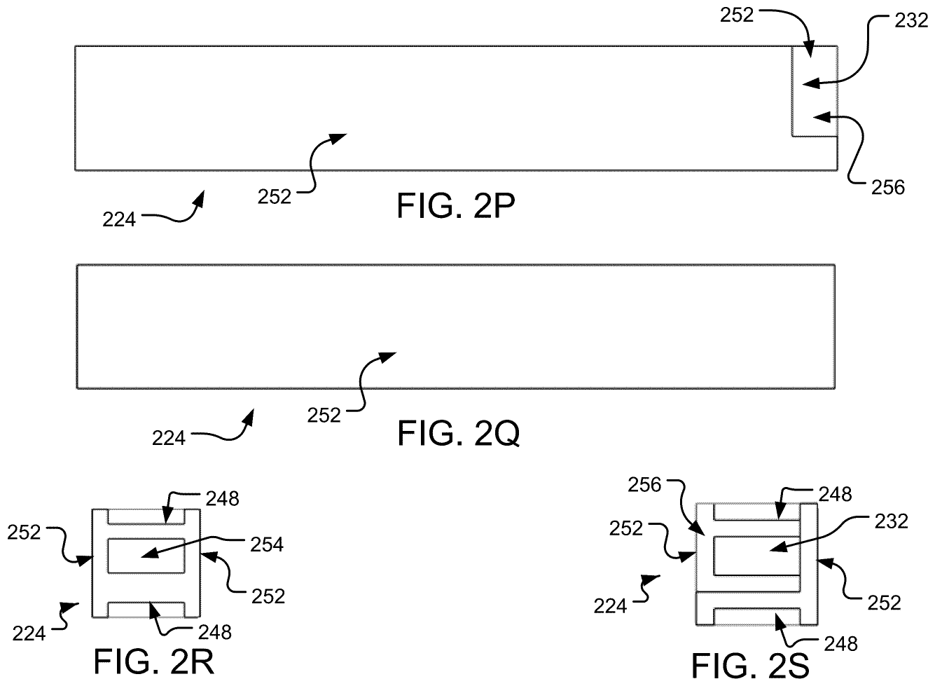

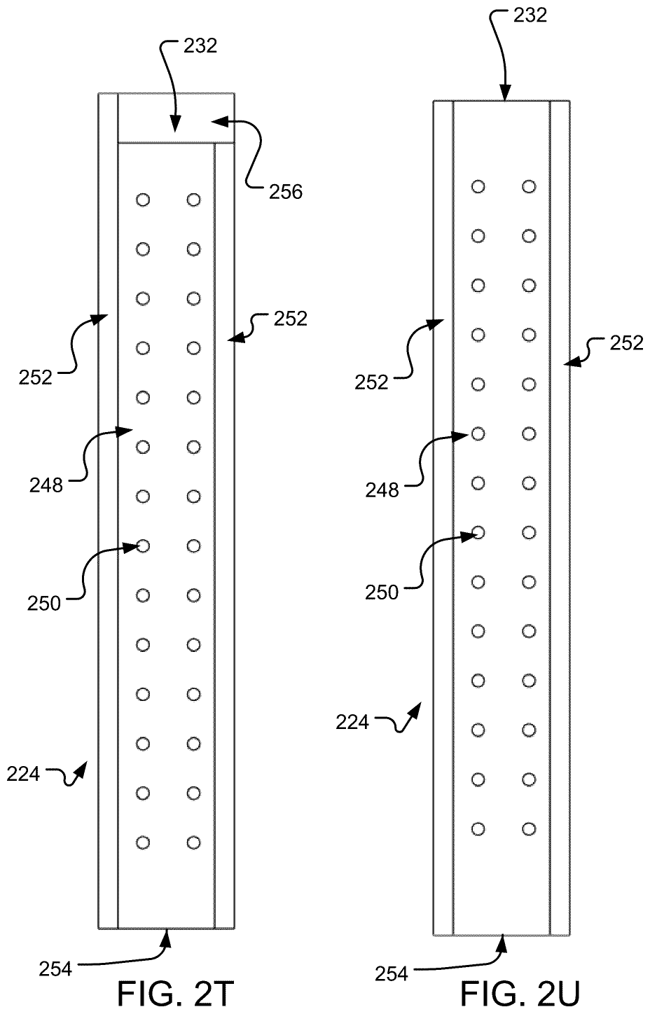

Embodiments of cellular transplant carrier devices and methods is further described with reference to FIGS. 2A-2U. The cellular transplant carrier device can be referred to as a cellular or cell substrate or cellular transplant carrier device. In this example, reference is made to FIG. 2A, which illustrates an overview of various stages in the production, transportation, and implantation of cellular transplants, such as hiPSC-RPE transplants. In certain embodiments, the cell transplant as referenced herein includes a plurality of cells, which may be present in layers (e.g., monolayer), or on tissue containing cells, or on a substrate.

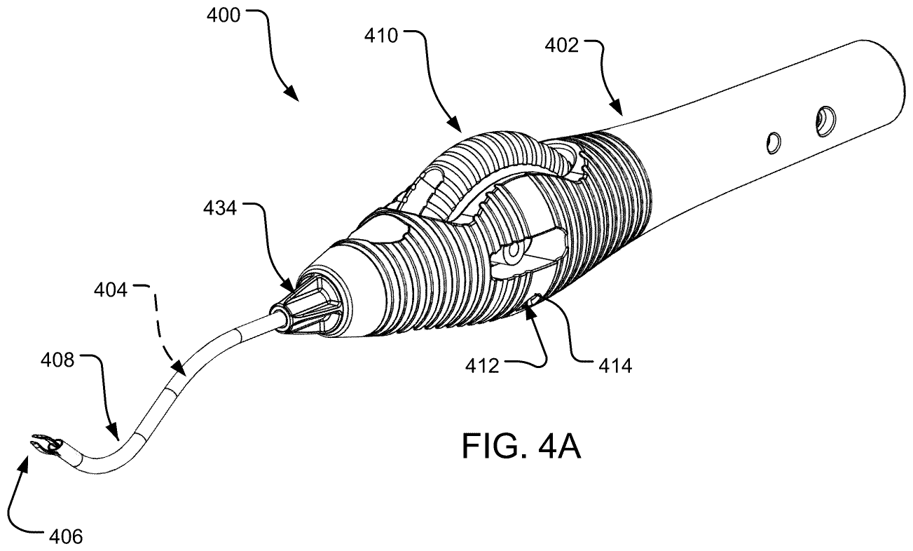

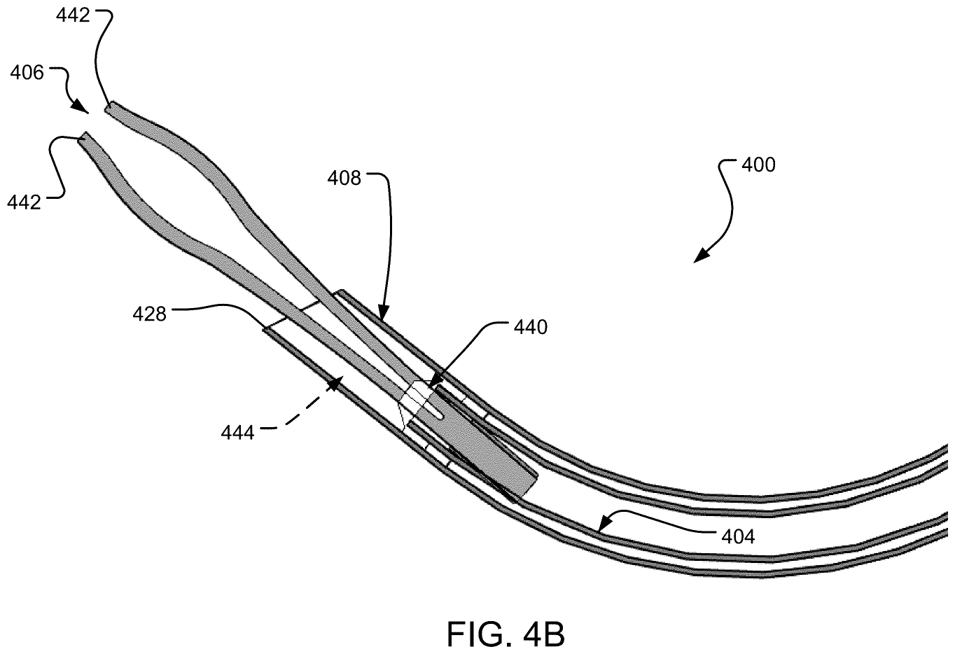

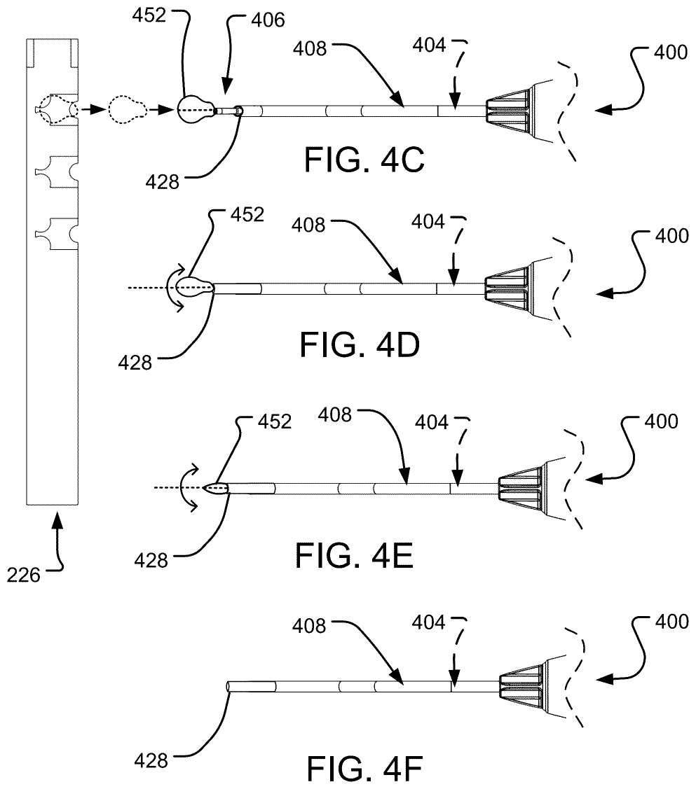

As observed in [block 200] of FIG. 2A, cell cultures 200 can be removed from a dish or tray and placed on a PET scaffold. From the removed cell cultures on a PET scaffold 202, illustrated in [block 202], a trephine or punch 204 (illustrated in further detail in FIG. 2B) can be used to obtain custom sized cell transplant (also referred to as cell implant) 206 (illustrated in further detail in FIG. 2C). As illustrated in [block 206], the cell transplants 206 are loaded into a cell carrier 208 (illustrated in further detail in FIGS. 2D-2U), which is designed to hold the cell transplants implants and/or substrates 206. The cell substrate carrier 208 can then be loaded into a carrier or chamber 210 (e.g., tube) for storage, transport or transportation to an operating room, as illustrated in [block 208]. Then, as illustrated in [block 210] of FIG. 2A, the cell carrier 208 can be removed from the chamber 210, and a delivery tool 212 (illustrated and described as a surgical implantation device 400 in FIGS. 4A-4F) can be utilized to obtain a cell transplant 206 from the carrier 208 and use it in a surgical procedure.

Turning to FIG. 2B, the trephine 204 is illustrated in a side view. The trephine 204 includes a handle and a distal cutting tip 214 that outlines the shape of the cell transplant 206. As illustrated in the figure, the distal cutting tip 214 includes a circular portion 216 illustrated to the right in the close-up image of FIG. 2B, and an asymmetrical tab portion 218 extending to the left of the circular portion. The tab 218 is asymmetrical in that it is not symmetric along the longitudinal axis (shown by dotted line). Instead, it includes a larger or prominent portion at the top and a smaller or inferior portion at the bottom.

FIG. 2C depicts a top view of the cell transplant or cell implant 206. The shape of the transplant 206 is defined by the cut made by the distal cutting tip 214 of the trephine 204 in FIG. 2B. Like the distal tip 214 of the trephine 204, the cellular transplant 206 has a circular portion 220 and an asymmetrical tab portion 222 that includes a larger portion (upper end of image) and a smaller portion (lower end of image). In this way, the orientation of the cellular transplant 206 is readily apparent (cell side up or upside down) by observing the asymmetrical tab portion 222 of the cellular transplant 206. For example, the cellular transplant 206 can be cell side up when the larger portion of the asymmetrical tab portion 222 is positioned in a clockwise direction from the smaller portion when the transplant 206 is viewed from above. This is an example, and the transplant 206 can be differently (e.g., oppositely) configured without limitation.

Exemplary dimensions for the cellular transplant 206 are illustrated in the figure, with a diameter of the circular portion being about 2 mm. A width of the tab portion 222 can be about 1.03 mm. A length of the tab portion 222 can be about 0.80 mm. These dimensions are exemplary for one embodiment of an iPSC-RPE (e.g.,hiPSC-RPE) cellular transplant. The dimensions can be modified given the type of implant and the biological environment for the transplant.

The tab portion 222 is configured to be grasped by a delivery instrument. The circular portion 220 is to be minimally disturbed during loading into the cell carrier 208, which is illustrated in FIG. 2D. In one example, the cell substrate or cellular transplant carrier 208 is illustrated in its component pieces, which include the carrier housing or chest 224 and the carrier plate or drawer 226. The carrier plate 226 includes a body 228 and chambers 230 defined in the body 228. Each of the chambers is sized and shaped to receive the cell transplant or cell implant 206 illustrate in FIG. 2C. When the cellular implant or cell transplant 206 is received in the chamber 230, the cellular transplant or cellular implant 206 is secured in place within the chamber 230 while being partially exposed to permit removal from the chamber 230, as is described subsequently. The carrier housing 224 includes a carrier plate opening 232 leading to an inner compartment configured to removably receive the carrier plate 226. In accordance with this embodiment regarding the carrier plate 226 illustrated in FIG. 2D, the plate 226 includes three chambers 230. However, the plate 226 can include more or less chambers 230 then illustrated. For example, the plate 226 can include a single chamber 230. In a certain examples, the plate 226 can include two chambers 230. In another example, the plate 226 can include four chambers 230. In a yet another example, the plate 226 can include five chambers 230. In one example, the plate 226 can include more than five chambers 230.

FIG. 2E is a close-up top view of a chamber 230 of the carrier plate 226 with a cell transplant or cellular substrate or cellular implant 206 shown in dotted line which is positioned within the chamber 230. As observed in the figure, the body 228 of the carrier plate 226 includes an overhanging structure made up of a pair of tabs 232. The overhanging structure defined by the tabs 232 provides a slot (not visible in FIG. 2E) underneath the tabs 232. The slot is sized and shaped to retain a portion or percentage of the cell transplant from migrating from the chamber 230 when, for example, the carrier plate 226 is positioned within the carrier housing (not shown) and the entire cell carrier 208 is positioned in a tube with solution (e.g., preservation medium or stabilizing composition) therein. As can be seen by the dotted line of the cell transplant or cellular implant 206, a portion of the circular portion of the cell transplant can fit within the slot and underneath the overhanging structure of the tabs 232.

As illustrated in FIG. 2E, the chambers are defined on a side edge 234 of the carrier plate 226. The chamber 230 further includes a recessed portion 236 defined on the side edge 234 of the carrier plate 226. The tab portion of the cell transplant 206 could overhang the recessed portion 236 when the cell transplant 206 is received within the chamber 230. The recessed portion 236 permits a gripping portion of a surgical instrument to grasp the cell transplant 206 by the tab portion and remove it from the carrier plate 226 with minimal disruption of the cells.

FIGS. 2F-2M depict various images of the carrier plate 226. For example, FIGS. 2F-2M depict, respectively, an isometric top view, an isometric side view, a chamber side view, an opposite side view, a first end view, a second end view, a bottom view, and a top view of the carrier plate 226. These figures are described together with specific reference to certain figures, where appropriate.

In FIGS. 2F and 2G, the chamber 230 of the carrier plate 226 is illustrated in detail. For instance, the figures illustrate a slot 238 formed underneath the overhanging structure formed by the tabs 232 of the body 228 of the carrier plate 226. The slot 238 extends underneath the tabs 232 such that the tabs 232 support the cell transplant in place within the chamber 230 of the carrier plate 226. The recessed portion 236 is defined on the side edge 234 of the carrier plate 226 and includes a partial cylindrical divot that extends into the side edge 234. The chamber 230 includes a planar surface 240 for supporting the cell transplant 206 thereon. The planar surface 240 is recessed from a top surface 242 of the body 228 of the carrier plate 226. The planar surface 240 is flanked by opposing sidewalls 244.

As illustrated in the figures, the carrier plate 226 includes stop structure 246 at an end thereof. The stop structure 246 includes a pair of structures extending upwards from the top surface 242 of the body 228 of the plate 226. The stop structure 246 contacts the carrier housing when positioned therein to prevent further advancement in the inner compartment of the housing.

The chamber 230 is generally exposed to expose the cell transplant 206 from the top and a side of the carrier plate 226. As such, a cell transplant or cell transplant 206 positioned within the chamber 230 can be removed from a lateral direction as indicated by the arrow in FIG. 2M.

The carrier housing 224 is illustrated in FIGS. 2N-2U. FIGS. 2N-2U depict various images of the carrier housing 224. For example, FIGS. 2N-2U depict, respectively, a first isometric top view, a second isometric top view from an opposite side, a side view, an opposite side view, a first end view, a second end view, a top view, and a bottom view of the carrier housing 224. The figures are described together with specific reference to certain figures, where appropriate.

As illustrated in the figures, the carrier housing 224 includes a pair of walls 248 (top and bottom walls) having holes 250 defined therein. The walls 248 extend the length of the housing 224 and oppose each other. The carrier housing 224 further includes a pair of side walls 252 that oppose each other and that form adjacent walls to the pair of walls 248 with the holes 250. The side walls 252 do not include through holes defined therein; however, in certain instances, the side walls 252 can include holes. The holes 250 defined in the top and bottom walls 248 permit the passage of fluid into and out of the inner compartment. At an insertion end of the carrier housing 224 is the carrier plate opening 232 in which leads to the inner compartment, which is sized and shaped to removably receive the carrier plate 226 (not shown). At the opposite end of the carrier housing 224, the inner compartment daylights at another opening 254 that further permits the passage of fluid through the carrier housing 224 and the carrier plate 226 when positioned therein.

In one example, the top wall 248 of the carrier housing 224 is shorter than the bottom wall 248. In addition, one of the side walls 252 is shorter than the other wall 252. In this way, the carrier housing 224 defines an access area 256 for grasping the carrier plate 226 when it is positioned in the inner compartment of the carrier housing 224. For example, when the carrier plate 226 is positioned within the inner compartment of the carrier housing 224, the stop structure 246 prevents the carrier plate 226 from extending farther into the inner compartment and the stop structure 246 can be positioned within the access area 256 for ease of inserting and removing the carrier plate 226.

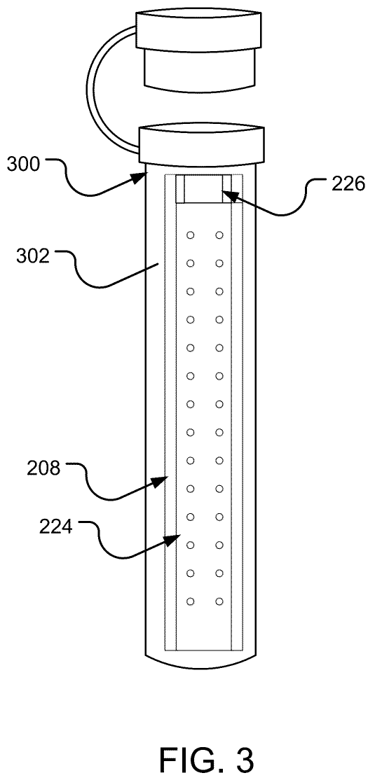

FIG. 3 depicts the cell transplant carrier 208 positioned in a chamber 300 such as a sealable tube. The chamber 300 includes a fluid 302 therein such as a preservation medium. The cell carrier 208 includes the carrier housing 224 and carrier plate 226 positioned therein, where the carrier plate 226 is supporting one or more cell transplants 206 (not visible in FIG. 3) therein. The chamber 300 may be shipped or transported in this orientation. In certain instances, the cell carrier 208 is sized and shaped to fit within a standard 2.0 mL tube.

FIG. 4A depicts an isometric view of an exemplary surgical implantation device 400 useable for retrieving a cell transplant 206 from a carrier plate 226. As illustrated in the figure, the surgical implantation device 400 includes a handpiece 402, a shaft 404, a forceps or micro-jaws 406 at the distal end of the shaft 404, a tubular sheath 408 that slides over the shaft 404 and forceps 406, and an actuator or wheel 410 that is moveably coupled to the handpiece 402 via a pin 412. In certain examples, the pin 412 can include a spring that biases the pin 412 in a certain direction. For instance, the pin 412 can bias the actuator 410 in a distal position with the tubular sheath 408 positioned over the forceps 406. In certain instances, the pin 412 can bias the actuator 410 in the opposite, proximal direction. The actuator 410 is pivotable about the pin 412 which is also received within a transverse opening 414 in the handpiece 402.

FIG. 4B depicts an up-close cross-sectional side view of the surgical implantation device 400. As illustrated in the figure, the device 400 is in a deployed state. That is, distal tips 442 of the forceps 406 are positioned outside a distal opening 428 of the tubular sheath 408. In this state, the forceps 406 are opened and designed to release its grasp on the cellular transplant (not shown). As illustrated in the figure, the distal tips 442 are not touching in the deployed state. Also as illustrated in the figure, the forceps 406 are coupled to a distal end of the shaft 404 and a seal 440 is positioned between the shaft 404 and the inner wall of the tubular sheath 408 to prevent backflow of fluid into the device 400 when this portion of the device 400 is in a pressurized environment (e.g., eye).

In another example, a non-deployed state of surgical implantation device 400 would be when the distal tips 442 of the forceps 406 are positioned within the lumen (inside of the tubular sheath 408) 444 of the tubular sheath 408. Distal advancement of the tubular sheath 408 relative to the shaft 404 and the forceps 406 can cause the device 400 to transition from a deployed state to a non-deployed state. Conversely, proximal retraction of the tubular sheath 408 relative to the shaft 404 and the forceps 406 would cause the device 400 to transition from a non-deployed state to a deployed state. In the non-deployed state, the distal tips 442 of the forceps 406 would be forced together via compression by the inner walls of the tubular sheath 408. In this state, the forceps 406 would grasp the cellular transplant (not shown) and house the same within the lumen 444 of the tubular sheath 408. In this manner, the only way to employ the grasping and releasing of the forceps 406 is via movement of the tubular sheath 408 relative to the shaft 404.

FIGS. 4C-4F illustrate the grasping of a cell transplant 452 by the forceps 406 of the surgical implantation device 400 from a carrier plate 226 and the subsequent housing of the cell transplant 452 in the lumen of the tubular sheath 408. FIG. 4C depicts a top view of the device 400 grasping a cell transplant 452 having a shape as illustrated in FIG. 2C. As illustrated in the figure, the forceps 406 are extended out from the distal opening 428 of the tubular sheath 408. The forceps 406 are closed just enough to grasp the cell transplant 452 by the tubular sheath 408 distally advancing relative to the shaft 404 and forceps 406, which remain stationary. As illustrated in this figure, the cell transplant 452 was initially housed in a cell chamber of a cell carrier 226.

As illustrated in FIG. 4D, the tubular sheath 408 is beginning to distally advance relative to the shaft 404 and the forceps 406. In doing so, the cell transplant 452 comes into contact with the beveled distal opening 428 of the tubular sheath 408. The cell transplant 452 begins to concavely fold along the dotted longitudinal axis. That is, the cell transplant 452 begins to conform to a tube, matching a shape of the inner walls of the tubular sheath 408. FIG. 4E illustrates further distal advancement of the tubular sheath 408 and further folding of the cell transplant 452 into a tube-shape. Finally, FIG. 4F illustrates the cell transplant being fully housed within the tubular sheath 408. This figure illustrates the non-deployed state of the device 400 and cell transplant.

With the cell transplant loaded into the device 400, the device can be utilized in a surgical procedure. In order to transition the device 400 from the non-deployed state to the deployed state, the steps illustrated in FIGS. 4C-4F can be reversed. For example, the tubular sheath 408 can be proximally retracted relative to the forceps 406 and shaft 404, which will expose the cell transplant 452 out of the distal opening 428 of the tubular sheath 408. This causes the cell transplant 452 to unfold from a cylindrical shape to a generally flat shape or orientation.

### EXAMPLES

It is understood that the examples and embodiments described herein are for illustrative purposes only and are not intended to limit the scope of the claimed invention. It is also understood that various modifications or changes in light of the examples and embodiments described herein will be suggested to persons skilled in the art and are to be included within the spirit and purview of this application and scope of the appended claims.

Exemplary devices disclosed herein provide solutions to preventing, reducing the risk of, and treating currently incurable eye disorders. Certain exemplary devices disclosed herein provide solutions to preventing, reducing the risk of, and treating retinal degenerative conditions where transplantation of retinal-directed cell transplants is one therapy. These devices and methods apply to pre-clinical and clinical phases of cell-based regenerative therapies. In certain disclosure related to devices herein, a transplantation device is disclosed that is the first to provide an all-in-one foldable technology that for example, minimizes incision size, controlled delivery speed, no fluid reflux, curved translucent tip, usability of loading and in vivo reloading, and ergonomic handle. This transplantation device can be combined with a customizable transplant carrier device disclosed herein to ensure viability of cell transplants during storage and/or transport, proper orientation of cell transplant, and usability of loading into the transplantation device.

In another example, a customizable transplant carrier device is designed to ensure viability of cell transplant during storage and transport, proper orientation of cell transplant, and usability of loading into a transplantation device. This transplantation delivery device is the first to provide all-in-one foldable technology that minimizes incision size, controls delivery speed, reduced to no fluid reflux, curved translucent tip, usability of loading and in vivo reloading, having an ergonomic handle. Combined these two technologies offer an all-encompassing surgical kit for implantation of cell-based substrates and transplants to an eye of a subject.

The terms and expressions which have been employed herein are used as terms of description and not of limitation, and there is no intention in the use of such terms and expressions of excluding any equivalents of the features shown and described or portions thereof, but it is recognized that various modifications are possible within the scope of the invention claimed. Therefore, it should be understood that although the present invention has been specifically disclosed by some embodiments, exemplary embodiments and optional features, modification and variation of the concepts herein disclosed may be resorted to by those skilled in the art, and that such modifications and variations are considered to be within the scope of this invention as defined by the appended claims. The specific embodiments provided herein are examples of useful embodiments of the present invention and it will be apparent to one skilled in the art that the present invention may be carried out using variations of the devices, device components, methods steps set forth in the present description. As will be obvious to one of skill in the art, methods, and devices useful for the present methods can include optional composition and processing elements and steps.

What is claimed:

1. A cell transplant carrier comprising:
a carrier plate comprising a body and one or more chambers defined in the body, wherein each of the one or more chambers is configured to receive a cell transplant therein; and
a carrier housing comprising a carrier plate opening leading to an inner compartment configured to removably receive the carrier plate.

2. The carrier of claim 1, wherein the carrier plate is rectangular in shape.

3. The carrier of claim 1, wherein each of the one or more chambers includes a slot portion defining an overhang of the body of the carrier plate, the slot portion configured to retain the cell transplant from migrating therefrom.

4. The carrier of claim 3, wherein each of the one or more chambers extends into a side edge of the carrier plate.

5. The carrier of claim 4, wherein each of the one or more chambers includes a recessed portion defined on the side edge of the carrier plate, the cell transplant including a tab portion configured to overhang the recessed portion when the cell transplant is received within the one or more chambers.

6. The carrier of claim 1, wherein the one or more chambers are symmetrically distributed along the dimensions of the drawer.

7. The carrier of claim 1, wherein the carrier housing includes a plurality of openings extending from an outer surface to the inner compartment to provide a plurality of passageways for a preservation fluid to pass therethrough.

8. The carrier of claim 7, wherein the carrier housing comprises four sidewalls, the plurality of passageways defined on at least two of the sidewalls.

9. The carrier of claim 8, wherein, in an assembled state, the carrier plate is positioned within the inner compartment of the carrier housing and the tab portion of the cell transplant opposes one of the sidewalls of the carrier housing without the plurality of passageways defined therein.

10. The carrier of claim 1, wherein the carrier housing and the carrier plate, when positioned within the inner compartment of the carrier housing, are sized to fit within a tube of predetermined size for storage and/or transport.

11. The carrier of claim 1, wherein each of the one or more chambers comprises two symmetrical flat tabs configured to hold the cell transplant in place in a correct orientation within each chamber.

12. The carrier of claim 1, wherein at least one of the one or more chambers has a length of about 2 mm to about 20 mm, a width of at least 1 mm and not more than 3 mm, and a height of at least 0.25 mm and not more than 0.75 mm.

13. The carrier of claim 1, wherein the carrier housing has a cuboid shape.

14. The carrier of claim 1, wherein the carrier housing and/or carrier plate is 3D printed.

15. The carrier of claim 1, wherein the carrier housing and/or the carrier plate is manufactured from at least one of VeroClear, Med610, Med615, and Med620.

16. The carrier of claim 1, wherein the cell transplant comprises a retinal pigment epithelium (RPE) cell transplant.

17. The carrier of claim 1, wherein the cell transplant comprises a neural retina-RPE cellular transplant.

18. The carrier of claim 1, wherein the cell transplant comprises a cellular transplant including retinal cells.

19. The carrier of claim 1, wherein each of the one or more chambers is shaped to cause the cell transplant to be at least partially exposed or accessible in order to permit removal of the cell transplant therefrom.

20. The carrier of claim 1, wherein the cell transplant comprises a substrate and a plurality of retinal pigment epithelium (RPE) cells on the substrate.

21. A carrier device comprising:
a cell transplant carrier according to any one of claims 1-20;
one or more cell transplants housed within the cell transplant carrier; and
a chamber configured to receive the cell transplant carrier therein.

22. The carrier device of claim 21, further comprising a medium or stabilizing composition configured to be received within the chamber.

23. The carrier device of claim 21, further comprising a temperature controller configured to control a temperature of the medium or stabilizing composition.

24. The carrier device of claim 21, further comprising an oxygen controller configured to control oxygen levels of the medium or stabilizing composition.

25. The carrier device of claim 21, further comprising a C02 controller configured to control pH of the medium or stabilizing composition.

26. A method of preserving viability of cells or a cellular implant or cellular tissue, comprising:
placing the cells or a cellular implant or cellular tissue into a cell transplant carrier according to any one of claims 1-20;
transferring the cell transplant carrier into a chamber configured to receive the cell transplant carrier to form a carrier device; and
filling the carrier device with a stabilizing medium and preserving the viability of the cells or cellular implant or cellular tissue.

27. The method of claim 26, further comprising removing the cells or the cellular implant or cellular tissue from the cell transplant carrier using a surgical device.

28. The method of claim 27, further comprising implanting the cells or the cellular implant or cellular tissue into an eye of a subject using the surgical device and treating an eye condition in the subject.

29. The method of claim 26, wherein the eye condition comprises a condition of the retina.

30. A surgical kit comprising:
a cell transplant carrier of any one of claims 1-20;
one or more cell transplants housed within the cell transport carrier; and
instructions for use.

31. The surgical kit of claim 30, further comprising a trephine configured to cut the cell transplant.

32. The surgical kit of claim 31, wherein the trephine is configured to cut the cell transplant in a shape comprising a circular portion and a tab portion extending outward from a perimeter of the circular portion.

33. The surgical kit of claim 30, further comprising a surgical implantation device configured to releasably grasp the cell transplant.
